(19) United States
(12) Patent Application Publication
(10) Pub. No.: US 2015/0063538 A1
(43) Pub. Date: Mar. 5, 2015

(54) SYSTEM AND METHOD OF X-RAY RADIATION REDUCTION

(71) Applicant: Elmer Stevenson, Winterville, NC (US)

(72) Inventor: Elmer Stevenson, Winterville, NC (US)

(21) Appl. No.: 14/010,847

(22) Filed: Aug. 27, 2013

(51) Int. Cl. A61B 6/10; A61B 6/06

(52) U.S. Cl. CPC A61B 6/10; A61B 6/06
USPC 378/55

(57) ABSTRACT

A system and method of reducing radiation exposure during an X-ray procedure comprising the steps of: inserting an automated collimation device onto an X-Ray machine; implementing infrared or ultrasonic measuring features into the collimation device; measuring the distance of the X-ray source to the anatomy of a patient; measuring the thickness of the anatomy of the patient; and automatically adjusting the distance and degree of radiation emission based upon the results of distance and thickness measurements.

### BACKGROUND OF THE INVENTION

1. Field of Invention

The present invention relates to a system and method to reduce radiation exposure during the performance of an X-ray on an individual.

2. Description of Related Art

One well-known aspect of medical imaging involves the use of X-rays and X-ray apparatuses. During the X-ray the individual is placed under a X-ray source and an image is taken using radiation that is emitted from the X-ray source to provide a image of the underlying bones and tissue of an individual. X-ray radiation however must be limited to avoid any health risks to the patient. Dosage rates are regulated and these rates must be kept below the maximum exposure rate to ensure safe operation on patients. Further a particular distance must be maintained between the subject anatomy and the X-ray source in order not to exceed a particular dosage rate of radiation and to achieve a satisfactory medical X-ray image. In a number of situations, the distance between the X-ray radiation and the patient has been fixed. Many of the existing X-ray apparatuses fail to consider the patient's anatomy that's being X-rayed.

Some techniques have been developed to vary the distance of the X-ray from the subject patient as a means to reduce the radiation exposure to the patient. Such a method is disclosed in U.S. Pat. No. 7,428,295 to Fehrer. The Fehrer patent discloses a method where the X-ray apparatus places a patient at a variable distance relative to the X-ray source, so the dose rate emitted is reduced given the reduction of the distance and/or increase given the increase of the distance. However one drawback to this variable distance is that the X-ray machine does not take into consideration the thickness of the anatomy that is being X-rayed. This aspect can provide a more adequate consideration of the amount of X-ray radiation exposure needed to have a satisfactory image. Therefore it would be advantageous to have a system or device installed on an existing X-ray machine that takes into consideration both distance and thickness of a subject's anatomy or executing the X-ray radiation.

### SUMMARY OF THE INVENTION

The present invention relates to a system and method of reducing radiation exposure during an X-ray procedure comprising the steps of: inserting an automated collimation device onto an X-Ray machine; implementing infrared or ultrasonic measuring features into the collimation device; measuring the distance of the X-ray source to the anatomy of a patient; measuring the thickness of the anatomy of the patient; and automatically adjusting the distance and degree of radiation emission based upon the results of distance and thickness measurements.

### BRIEF DESCRIPTION OF DRAWINGS

FIG. 1 depicts implementation of X-ray measuring system in accordance with the present invention.

### DETAILED DESCRIPTION

The present invention related to a system and method for measuring distance and anatomy thickness of a patient while undergoing an X-ray. This present invention helps to lower the radiation exposure as a means to reduce the risk of cancer, radiation burns, skin diseases and radiation poison that can occur due to prolonged exposure to X-rays. The present system automates the collimation process by measuring the distance and thickness of the particular anatomy that is undergoing the X-ray procedure. The present invention implements a system that measures both the distance from the patient to the X-ray machine and also the thickness of the patient's anatomy so that the radiation exposure is kept at a minimum considering both factors. The present invention eliminates the need to manually collimate before performing X-ray procedures. The present invention uses an infrared or ultrasonic device and measures the distance the patient is away from the X-ray machine and further performs a measurement of a thickness of the body part before the procedure starts. This present invention prevents the overexposure of radiation and eliminates the need to rotate the machine in certain instances.

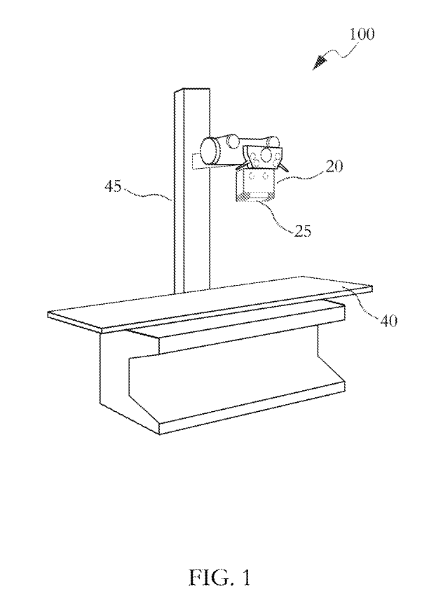

In reference to FIG. 1, an X-ray Machine 100 is depicted. The X-ray machine 100 includes a Table 40 and Pedestal 45. The X-ray Machine 20 is attached to the Pedestal 45 and may be adjusted vertically along the vertical length of the Pedestal 45. Installed in the X-ray Machine 20 is an automated collimation device 25. The collimation device 25 measures both distance from the X-ray Machine 20 to the patient that may be on the Table 40 and the overall thickness of the particular anatomy that is being X-rayed by the X-ray machine. These calculations are provided to provide an optimum distance for the X-ray machine considering both factors of distance and thickness of the anatomy. Once these measurements are taken and calculations are provided the X-ray may be performed with the minimum amount of radiation exposure.

The use of the automated collimation Device 25 creates an optimized situation for conducting X-rays. The automated collimation device 25 includes the use of infrared or ultrasonic rays to execute the measuring functions. Further individuals may experience less exposure to the radiation rays and thus reduce the risks that are associated with X-rays. The instant invention has been shown and described in what it considers to be the most practical and preferred embodiments. It is recognized, however, that departures may be made there from within the scope of the invention and that obvious modifications will occur to a person skilled in the art.

What is claimed is:

1. A method of reducing radiation exposure during an X-ray procedure comprising the steps of:
a. inserting an automated collimation device onto an X-Ray machine;
b. implementing infrared or ultrasonic measuring features into the collimation device;
c. measuring the distance of the X-ray source to the anatomy of a patient;
d. measuring the thickness of the anatomy of the patient; and
e. automatically adjusting the distance and degree of radiation emission based upon the results of steps c and d.

2. A system of reducing radiation exposure during an X-ray procedure comprising:
a. an automated collimation device placed on an X-ray machine;
b. a vertically adjustable X-ray apparatus;
c. infrared or ultrasonic measuring features within the automated collimation device, where the X-ray source is vertically adjusted based a vertical measurement distance to a patient and a thickness of the patient's anatomy.
